(19) United States
(12) Patent Application Publication
(10) Pub. No.: US 2012/0029204 A1
(43) Pub. Date: Feb. 2, 2012

(54) SYNTHESIS OF SUBSTITUTED-3-AMINOPYRAZOLES

(75) Inventors: Carmen S. Alvarez, Livingston, NJ (US); Michael P. Dwyer, Scotch Plains, NJ (US); Timothy J. Guzi, Sudbury, MA (US); Kartik M. Keertikar, East Windsor, NJ (US); Marc A. Labroli, Moorestown, NJ (US); Kamil Paruch, Tisnov (CZ); Cory Seth Poker, South Plainfield, NJ (US)

(73) Assignee: Schering Corporation, Kenilworth,, NJ (US)

(21) Appl. No.: 12/602,716

(22) PCT Filed: Jun. 4, 2008
(86) PCT No.: PCT/US08/07009

§ 371 Date: Aug. 11, 2011

Related U.S. Application Data
(60) Provisional application No. 60/942,558, filed on Jun. 7, 2007.

(51) Int. Cl. C07D 403/04

(52) U.S. Cl. 548/365.4

(57) ABSTRACT

The present invention discloses a process of preparing compound of formula (I): wherein A, M, and Z are as defined herein. An example of a compound of formula (I) is 3-amino-1-methyl-1H-1′H-4,4′-bispyrazole.

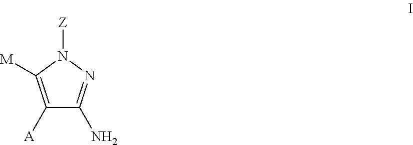

### FIELD OF THE INVENTION

This application discloses a novel process to synthesize certain substituted-3-aminopyrazoles, in general, and 3-amino-1-methyl-1H-1′H-4,4′-bispyrazole, in particular, and intermediates therefore.

### BACKGROUND OF THE INVENTION

3-Amino-1-methyl-1H-1′H-4,4′-bispyrazole (formula IA), is disclosed in U.S. patent application Ser. No. 11/245,401, published as US 2006/0128725 on Jun. 15, 2006, which is incorporated herein by reference.

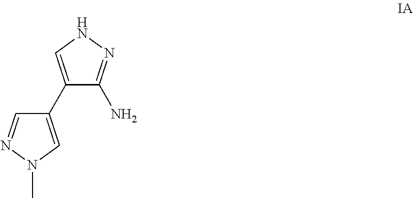

The compound of formula IA can be utilized as an intermediate in the synthesis of compounds of formula X:

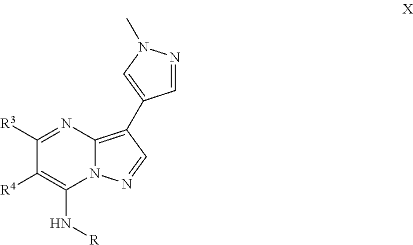

wherein R, R³, and R⁴ have the definitions described in the above-referenced US 206/0128725.

The compounds of formula X are useful as protein kinase inhibitors and can be useful in the treatment and prevention of proliferative diseases, for example, cancer, inflammation and arthritis. They may also be useful in the treatment of neurodegenerative diseases such as Alzheimer's disease, cardiovascular diseases, viral diseases and fungal diseases. Patent application Ser. No. 11/245,401, also teaches a method of making the compound of formula IA, and from IA compounds of formula X.

In view of the importance of protein kinase inhibitors, new, novel methods of making such compounds are always of interest.

### SUMMARY OF THE INVENTION

In one embodiment, the present application teaches a process of making the compound of formula IA, and more generally, a 3-aminopyrazole compound of formula I:

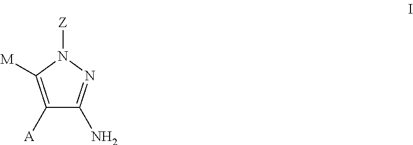

comprising:

(a) Converting a compound of formula II

A-H II

to a compound of formula III

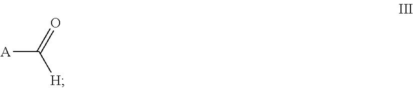

and either:

(b)(1) reacting the compound of formula III with the anion of tosylmethylisocyanide (TosMIC) to yield a compound of formula IV

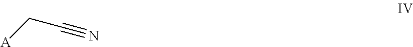

or:

(b)(2) Reducing the compound of formula III to the compound of formula V,

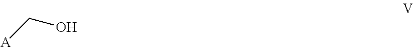

followed by conversion of the compound of formula V to the compound of formula IV; and thereafter:

(c) Acylating the compound of formula IV to a compound of formula VI;

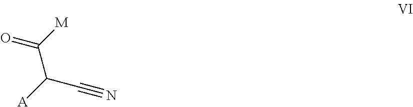

and

(d) Treating the compound of formula VI with a compound of formula VII,

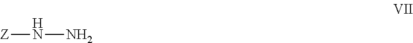

or one of its salts or hydrates, to yield the compound of formula I;

wherein:

A is selected from the group consisting of alkyl, cycloalkyl, aryl, and heteroaryl, each of which is independently unsubstituted or substituted with at least one W moiety;

M and Z are independently selected from the group consisting of H, alkyl, aralkyl, cycloalkyl, heterocyclyl, aryl and heteroaryl wherein each of said alkyl, aralkyl, cycloalkyl, heterocyclyl, aryl and heteroaryl is independently unsubstituted or substituted with at least one W moiety;

W is selected from the group consisting of alkyl, halo, cycloalkyl, heterocyclyl, aryl and heteroaryl.

The inventive process to make the compound of formula I or IA has several advantages over the previously disclosed process in US 2006/0128725: it is more economical, can be easily scaled-up and has flexibility with respect to varying the nature of the substituents A, M and Z, allowing application to the synthesis of compounds of the formula I. Thus, by varying the structure of the formyl compound of the formula III, differently substituted 3-aminopyrazoles may be produced with relative ease. Thus, for example, a non-limiting list of suitable formylated compounds of the formula III and the obtainable pyrazole compounds of the formula I, is shown in Table I:

TABLE 1

| Compounds of Formula III | Compounds of Formula I |
|---|---|
| 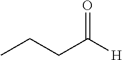 | 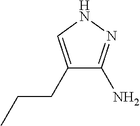 |
| 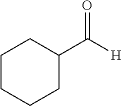 | 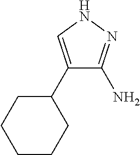 |
| 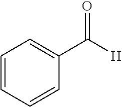 | 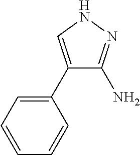 |
| 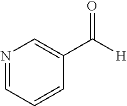 | 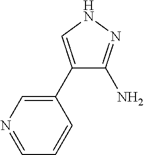 |
| 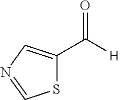 | 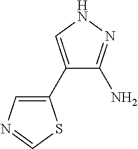 |

### DESCRIPTION OF THE INVENTION

In one embodiment, the present invention discloses a novel, easy-to-use process for preparing the compound of formula I.

As used above, and throughout the specification, the following terms, unless otherwise indicated, shall be understood to have the following meanings:

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and tert-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Y₁Y₂N—, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

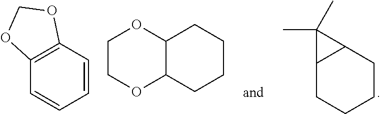

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” means a non-aromatic saturated monocyclic or multicyclic ring system comprising about 3 to about 10 ring atoms, preferably about 5 to is about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like.

“Heterocyclyl” may also mean a single moiety (e.g., carbonyl) which simultaneously replaces two available hydrogens on the same carbon atom on a ring system. Example of such moiety is pyrrolidone:

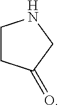

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

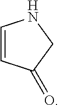

“Heterocyclenylalkyl” means a heterocyclenyl moiety as defined above linked via an alkyl moiety (defined above) to a parent core.

It should be noted that in heterocyclyl or heterocyclenyl ring systems (i.e., non-aromatic hetero-atom containing ring systems) of this invention, there are no hydroxyl groups on carbon atoms adjacent to a N, O or S, as well as there are no N or S groups on carbon adjacent to another heteroatom. Thus, for example, in the ring:

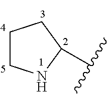

there is no —OH attached directly to carbons marked 2 and 5.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an intermediate.

The term “optionally substituted” means optional substitution with the specified groups, radicals or moieties.

As set forth above, any —NH in a heterocyclyl ring may exist protected such as, for example, as an —N(Boc), —N(CBz), —N(Tos) group and the like; such protections are also considered part of this invention. Similarly, any —NH, OH, or carbonyl substituent that is part of any group “A”, “M”, and “Z” as defined herein, may exist in a protected form. Such protections are also considered part of this invention. Some non-limiting examples include carbamates (protecting group for —NH), silyl ether (protecting group for OH), acetal/ketal (protecting group for carbonyls), as well as other protecting groups known to one of ordinary skill in the art.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

As set forth in the “Summary of the Invention” section, the present invention provides a process of preparing a compound of formula I:

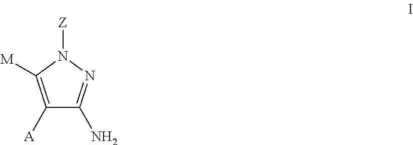

comprising:

(a) Converting a compound of formula II

A-H II

to a compound of formula III

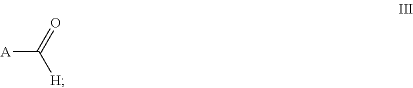

and either:

(b)(1) reacting the compound of formula III with the anion of tosylmethylisocyanide (TosMIC) to yield a compound of formula IV

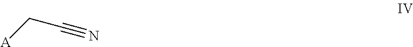

or:

(b)(2) Reducing the compound of formula III to the compound of formula V,

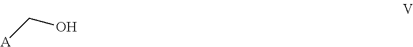

followed by conversion of the compound of formula V to the compound of formula IV; and thereafter:

(c) Acylating the compound of formula IV to a compound of formula VI;

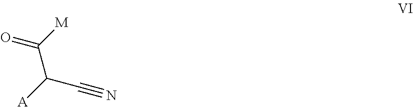

and

(d) Treating the compound of formula VI with a compound of formula VII,

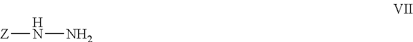

or one of its salts or hydrates, to yield the compound of formula I;

wherein:

A is selected from the group consisting of alkyl, cycloalkyl, aryl, and heteroaryl, each of which is independently unsubstituted or substituted with at least one W moiety;

M and Z are independently selected from the group consisting of H, alkyl, aralkyl, cycloalkyl, heterocyclyl, aryl and heteroaryl wherein each of said alkyl, aralkyl, cycloalkyl, heterocyclyl, aryl and heteroaryl is independently unsubstituted or substituted with at least one W moiety;

W is selected from the group consisting of alkyl, halo, cycloalkyl, heterocyclyl, aryl and heteroaryl.

In another embodiment, in formula I, A is heteroaryl.

In another embodiment, in formula I, A is 1-methyl-4-pyrazolyl.

In another embodiment, in formula I, M is H.

In another embodiment, in formula I, Z is H.

In another embodiment, the compound of formula I is:

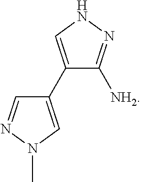

In another embodiment, in step (a), the conversion of the compound of formula II to the compound of formula III is carried out in the presence of a mixture of phosphorus oxychloride (POCl₃) and dimethylformamide.

In another embodiment, in step (b)(1), the anion of TosMIC is prepared by treating TosMIC with a basic compound.

In another embodiment, in step (b)(1), the anion of TosMIC is prepared by treating TosMIC with a basic compound, wherein said treatment is carried out at a temperature of about −78° C. to about −20° C.

In another embodiment, in step (b)(1), said basic compound is a metal hydroxide, metal oxide or metal alkoxide.

In another embodiment, in step (b)(1), said basic compound is a metal hydroxide, metal oxide or metal alkoxide, wherein said metal is an alkali or alkali earth metal.

In another embodiment, in step (b)(1), said basic compound is potassium tert-butoxide.

In another embodiment, in step (b)(1), said treatment is carried out in the presence of 1,2-dimethoxyethane (DME).

In another embodiment, in step (b)(1), the reaction of compound of formula III with the anion of TosMIC is carried out by stirring a mixture of the anion of TosMIC and the compound of formula III in a mixture of DME at a temperature of about −60° C. to about −50° C., followed by the addition of methanol, and heating of the mixture at a temperature of about 50° C. to the reflux temperature of the solvent mixture.

In another embodiment, in step (b)(2), the reduction of the compound of formula III to the compound of formula IV is carried out using sodium borohydride (NaBH₄).

In another embodiment, in step (b)(2), the conversion of the compound of formula V to the compound of formula IV is carried out in a two-step process involving in-situ conversion of the compound of formula V to the compound of formula V-A

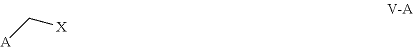

followed by conversion of the compound of formula V-A to the compound of formula IV, wherein in formula V-A, X is selected from the group consisting of halo, —S(O)₂alkyl, —S(O)₂aryl.

In another embodiment, in formula V-A, X is Cl.

In another embodiment, in step (b)(2), the conversion of the compound of formula V to the compound of formula IV is carried out in a two-step process involving in-situ conversion of the compound of formula V to the compound of formula V-A

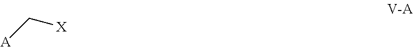

wherein in formula V-A, X is Cl, followed by conversion of the compound of formula V-A to the compound of formula IV, wherein the in-situ conversion of the compound of formula V to the compound of formula V-A is carried out using thionyl chloride (SOCl₂).

In another embodiment, in step (b)(2), the conversion of the compound of formula V to the compound of formula IV is carried out in a two-step process involving in-situ conversion of the compound of formula V to the compound of formula V-A

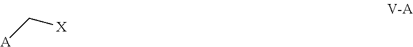

wherein in formula V-A, X is Cl, followed by conversion of the compound of formula V-A to the compound of formula IV, wherein the conversion of the compound of formula V-A to formula IV is carried out using tetraethylammonium cyanide.

In another embodiment, in step (c), the acylation of the compound of formula IV to the compound of formula VI is carried out using a basic compound and an acylating agent that is an alkyl ester of the acid MCOOH.

In another embodiment, in step (c), the acylation of the compound of formula IV to the compound of formula VI is carried out using a basic compound and an acylating agent that is an alkyl ester of the acid MCOOH, wherein said acylating agent is an ethyl, methyl, or isopropyl ester of formic, acetic, or benzoic acid.

In another embodiment, in step (c), the acylation of the compound of formula IV to the compound of formula VI is carried out using a basic compound and an acylating agent, wherein the acylating agent is ethyl formate.

In another embodiment, in step (c), the acylation of the compound of formula IV to the compound of formula VI is carried out using a basic compound and an acylating agent, wherein the basic compound is a metal hydroxide or metal alkoxide.

In another embodiment, in step (c), the acylation of the compound of formula IV to the compound of formula VI is carried out using a basic compound and an acylating agent, wherein the basic compound is potassium tert-butoxide.

In another embodiment, in step (d), the compound of formula VII is treated with a compound selected from the group consisting of hydrazine, hydrazine hydrate, and hydrazine salt.

In another embodiment, in step (d), the compound of formula VII is treated with a compound selected from the group consisting of hydrazine, hydrazine hydrate, and hydrazine salt, wherein the hydrazine salt is selected from the group consisting of hydrazine acetate, hydrazine hydrochloride, and hydrazine sulfate.

In another embodiment, in step (d), the compound of formula VI is to condensed with hydrazine monohydrochloride to yield the compound of formula I.

In another embodiment, the present inventive process is schematically described in Scheme I and Scheme II below:

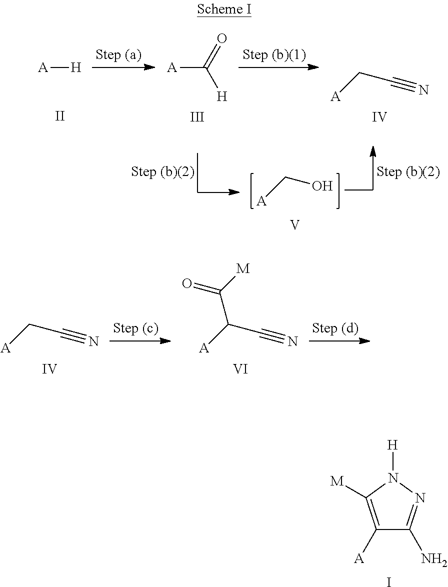

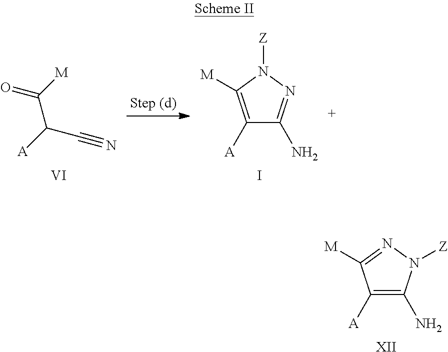

While the preferred reagents and reaction conditions for the various steps are described in detail in the Examples section, the following summarizes the details.

Where A is aryl or heteroaryl, the process may start with the aromatic or heteroaromatic compound of formula II, which is converted to the formyl compound of formula IV using formylation conditions known to those trained in the art. Non-limiting examples of this methodology include the use of a carbonyl source such as dimethylformamide, N-methyl-N-phenylformamide, or some other N,N′-disubstituted formamide in which the nitrogen may or may not be substituted with two of the same alkyl or aryl groups, in combination with an electrophilic reagent such as phosphorus oxychloride, trifluoromethanesulfonyl anhydride, phosgene, pyrophosphoryl chloride, or some other carboxylic or phosphoryl anhydride or halide. These two reagents, in equimolar amounts, or with an excess of one or the other, or both relative to the compound of formula II, can be mixed together and then added to the compound of formula II, or vice versa, or one of the reagents can be mixed with the compound of formula II followed by addition of the other reagent. The reaction may be conducted in one step or two, either or both of which may be conducted at a temperature ranging from 0° C. to the reflux temperature of the combined reagents. The reaction mixture is stirred at such temperature for about one hour or until the reaction is complete. The preferred method is the addition of the compound of formula II to a mixture of phosphorus oxychloride and dimethylformamide, both in equimolar amounts and in a 50% excess compared to the compound of formula II, at a temperature of 80° C. The formed product III may be purified or may be used crude after an aqueous work-up in the subsequent reaction step (b)(1) or (b)(2).

Alternatively, for the case where A is aryl or heteroaryl, and in the case where A is alkyl (including aralkyl), cycloalkyl, heterocyclyl, the process may start with the formyl compound of formula III. In the preferred method, the compound of formula III is dissolved in a suitable non-protic solvent and is then added to a solution, suspension or dispersion of the anion of tosylmethylisocyanide at a temperature of −78° C. to −20° C. A non-limiting list of suitable solvents includes dimethoxyethane, dioxane, tetrahydrofuran, diethyl ether, hexane, pentane, cyclohexane, cyclopentane, benzene, toluene, or some other ethereal or hydrocarbon solvent. Said anion is first prepared by adding a solution, suspension or dispersion of tosylmethylisocyanide to a solution, suspension or dispersion of an appropriate basic compound, both using the same suitable solvent and temperature as above, although the temperature used during preparation of the anion need not be the same as that used during the subsequent course of the reaction. A non-limiting list of appropriate basic compounds includes metal hydroxides, oxides or alkoxides wherein the metal is an alkali or alkaline earth metal. The preferred basic compound is potassium tert-butoxide. This mixture is stirred for about 1 to 3 hours, and then the resulting mixture is mixed with an alcoholic solvent, for example, methanol or ethanol, and is heated for a suitable period of time, generally 1 to 3 hours, at a temperature from 50° C. to the reflux temperature. The formed product IV may be purified or may be used crude after an aqueous work-up in the subsequent reaction step (c).

As an alternative to step (b)(1), the compound of formula III may be reduced to the alcohol compound of formula V and subsequently converted to the nitrile compound of formula IV. The reduction step may be carried out by dissolving the compound of formula III in a suitable solvent. A non-limiting list of suitable solvents includes ethanol, isopropanol, water, tetrahydrofuran, dioxane, diethyl ether, hexane, heptane, toluene, or benzene, and treating it with a reducing agent, suitably matched to the solvent, in such a way that is known to those trained in the art. Non-limiting examples of metal hydride reducing agents include sodium borohydride, lithium borohydride, lithium aluminum hydride, lithium triethylborohydride, lithium tri-sec-butylborohydride, sodium triacetoxyborohydride, sodium cyanoborohydride, zinc borohydride, and diisobutylaluminum hydride. Other suitable reducing agents include borane and substituted boranes such as 9-borabicyclononane. The reduction may be carried out over wide range of conditions of temperature, time and concentration depending on the choice of solvent and reducing agent. Reduction may also be accomplished using standard hydrogenation conditions involving the shaking or stirring of the compound of formula III in a suitable solvent in the presence of a transition metal catalyst and a hydrogen source. Non-limiting examples of catalysts include palladium, platinum and rhodium supported on generally accepted solid supports such as charcoal, or in the form of the free metal. Non-limiting examples of hydrogen sources include hydrogen gas, at atmospheric pressure or above, or transfer hydrogenation reagents such as formic acid, ammonium formate and cyclohexene. The formed product V may be purified or may be used crude after an aqueous work-up in the subsequent reaction of step (b)(2).

The alcoholic compound of formula V is converted to a suitable halide or sulfonate ester of formula V-A by treatment with a suitable reagent in a suitable solvent. A non-limiting list of suitable halides and sulfonate esters includes chlorides, bromides, iodides, and alkyl- and arylsulfonates including methanesulfonate, toluenesulfonate and nitrobenzenesulfonate. A non-limiting list of suitable reagents includes thionyl chloride, oxalyl chloride, phosphorus oxychloride, phosphorus trichloride, phosphorus pentachloride, hydrogen chloride, acetyl chloride, hydrogen bromide, phosphorus tribromide, phosphorus pentabromide, thionyl bromide, boron tribromide, trimethylsilyl iodide, triphenylphosphine-chlorine complex, triphenylphosphine and N-chlorosuccinimide, triphenylphosphine and bromine, triphenylphosphine and N-bromosuccinimide, triphenylphosphine and iodine, triphenylphosphine and N-iodosuccinimide, triphenylphosphine and carbon tetrachloride, triphenylphosphine and carbon tetrabromide, phosphorus and iodine, and any mixture of an alkyl- or arylsulfonyl halide and an organic base, such as 2,6-lutidine, pyridine, or 4-dimethylaminopyridine. A non-limiting list of suitable solvents includes dichloromethane, tetrachloromethane, chloroform, tetrahydrofuran, dioxane, dimethylformamide, pyridine, toluene, benzene, N-methylpyrrolidone and dimethylacetamide. The reaction is conducted at a temperature ranging from 0° C. to the reflux temperature of the solvent, and the reaction is generally stirred for 1 to 2 hours, or until it is complete. The preferred conditions are thionyl chloride and dichloromethane at 40° C. for one hour. After removing the excess reagent(s) and solvent by evaporation, or other means, the intermediate compound of formula V-A can be either purified, partially purified or used crude. The intermediate is then suspended, dissolved or dispersed in a suitable solvent and treated with an organic or inorganic cyanide reagent. A non-limiting list of suitable solvents includes acetonitrile, dimethylformamide, acetone, dimethylsulfoxide, dimethylacetamide, N-methypyrrolidone, dioxane, and tetrahydrofuran. A non-limiting list of suitable cyanide reagents includes tetraethylammonium cyanide, tetramethylammonium cyanide, tetrabutylammonium cyanide, sodium cyanide, and potassium cyanide. If the substituent A is a basic functionality, then the crude intermediate halide or sulfonate ester may be a salt, and if this is the case, a suitable organic or inorganic base may be added prior to the cyanide reagent. A non-limiting list of suitable bases includes triethylamine, diisopropylethylamine, N-methylmorpholine, N,N-dimethylaniline, imidazole, pyridine, sodium carbonate, potassium carbonate, and lithium carbonate. The reaction is stirred for 5 to 15 hours, or until the reaction is complete, at a temperature from room temperature to the reflux temperature of the solvent. The cyanide reagent and/or base may be used in a stoichiometric amount or in an excess, up to or exceeding 10 equivalents. The preferred method is to use triethylamine and tetraethylammonium cyanide in acetonitrile solvent at room temperature. The formed product IV may be purified or may be used crude after an aqueous work-up in the subsequent reaction step (c).

In step (c), the compound of formula IV, obtained either via step (b)(1) or step (b)(2), is acylated at the position adjacent to the nitrile functional group. The compound of formula IV is first dissolved, suspended or dispersed in a suitable solvent. It may then be added to a solution, suspension, or dispersion of a suitable base in a suitable solvent in the presence of a suitable acylating reagent. The acylating reagent may be added consecutively with the compound of formula IV, either in the same preparation or in a separate preparation, or may be added sequentially, either before or after addition of the compound of formula IV to the base. Both the acylating agent and the base may be used in any proportion from stoichiometric amounts to excesses up to five equivalents, or more. The reaction mixture is stirred at a temperature ranging from 0° C. to the reflux temperature of the solvent in an open or sealed system until the reaction is complete. A non-limiting list of suitable solvents include ethereal solvents such as dimethoxyethane, tetrahydrofuran, diethyl ether or dioxane, is alcoholic solvents such as methanol, ethanol or isopropanol, and hydrocarbon solvents such as hexane, pentane, toluene or benzene, depending on the chosen base. The base may be a preformed salt, such as a metal hydroxide or alkoxide, or may be formed in-situ by the reaction of an alkali metal or an alkali metal hydride with an alcoholic solvent or co-solvent to generate a metal alkoxide base. The acylating agent, chosen so as to incorporate the substituent group M into the pyrazole ring, may be any alkyl ester of the appropriate acid MCOOH. A non-limiting list of suitable acylating agents includes the ethyl, methyl, or isopropyl esters of formic acid, acetic acid, benzoic acid, and similar compounds. The preferred method is to add a solution of the compound of formula IV and the acyl ester in dimethoxyethane to a suspension of potassium tert-butoxide, followed by heating the resulting suspension 18 hours in a sealed pressure tube. The formed product VI is used after aqueous work-up in step 6.

In step (d), the compound of formula VI is dissolved, suspended or dispersed in a suitable solvent, such as ethanol, methanol or water, or a mixture of such solvents, and is treated with either hydrazine, hydrazine hydrate or an inorganic salt of hydrazine, such as hydrazine acetate, hydrazine hydrochloride or hydrazine sulfate (Scheme I), or with compound of formula VII, a similarly formed substituted derivative of hydrazine, such as methylhydrazine, ethylhydrazine, or phenylhydrazine (Scheme II). This step may be performed either with or without the addition of an inorganic or organic acid, such as acetic acid, hydrochloric acid or sulfuric acid, present in a molar amount equal to or exceeding the amount of hydrazine or the compound of formula VII. The resulting solution, suspension or dispersion is then heated at a temperature from 50° C. to the reflux temperature of the solvent until the reaction is complete. The preferred method is to treat the compound of formula VI with hydrazine monohydrochloride, or a substituted hydrazine monohydrochloride salt, in ethanol solution at a temperature of 90° C. When the reaction is performed using a hydrazine compound of formula VII, either the compound of formula I or the isomeric compound of formula XII, or a combination thereof, in any ratio, may be produced by the reaction. The formed product of formula I, and/or the product of formula XII, may then be isolated and purified by procedures well known to those skilled in the art, including extraction, crystallization and/or chromatographic purification.

In another embodiment, the present invention discloses a novel, easy-to-use process for preparing the compound of formula IA. The inventive process is schematically described in Scheme III:

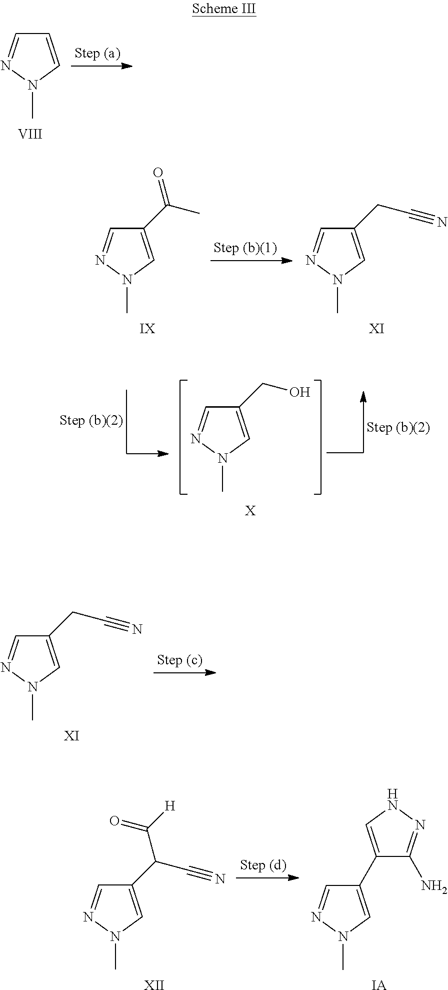

While the preferred reagents and reaction conditions for the various steps are described in detail in the Examples section, the following summarizes the details.

The process starts with the N-methylpyrazole compound of formula VIII, which is converted to the formyl compound of formula IX using formylation conditions known to those trained in the art, as described above for the compound of formula III.

The methods outlined above for steps (b)(1) or (b)(2) are then used to convert the compound of formula IX to the compound of formula XI. The compound of formula XI is then formylated according to the method outlined above for step (c) wherein M=H, and the acylating agent used is an ester of is formic acid. In the preferred method, this ester is ethyl formate. The resulting compound of formula XII is then treated with the compound of formula VII, wherein Z=H. The preferred method is to use hydrazine monohydrochloride in ethanol solution without any added acid. The formed product of formula IA may then be isolated and purified by procedures well known to those skilled in the art, including extraction, crystallization and/or chromatographic purification.

If desired, the compound of formula IA may be further converted to the protein kinase inhibitors of formula X by suitable procedures known to those skilled in the art.

The products of the various steps in the reaction schemes described to herein may be isolated and purified by conventional techniques such as, for example, filtration, recrystallization, solvent extraction, distillation, precipitation, sublimation and the like, well known to those skilled in the art. The products may be analyzed and/or checked for purity by conventional methods well known to those skilled in the art such as, for example, thin layer chromatography, NMR, HPLC, melting point, mass spectral analysis, elemental analysis and the like.

The following nonlimiting EXAMPLES are provided in order to further illustrate the present invention. It will be apparent to those skilled in the art that many modifications, variations and alterations to the present disclosure, both to materials, methods and reaction conditions, may be practiced. All such modifications, variations and alterations are intended to be within the spirit and scope of the present invention.

### EXAMPLES

Unless otherwise stated, the following abbreviations have the stated meanings in the Examples below:

### HPLC=High Performance Liquid Chromatography

NMR=nuclear magnetic resonance spectroscopy
DMSO=dimethylsulfoxide
DMF=dimethylformamide
DME=1,2-dimethoxyethane
mL=milliliters
g=grams
rt=room temperature (ambient)
TosMIC=tosylmethylisocyanide

### Example 1

### Preparation of Compound of Formula IX from the Compound of Formula VIII

Phosphorus oxychloride (6.92 g, 45.1 mmol, 1.50 equiv.) was cooled to 0° C. and then added drop-wise to anhydrous DMF (3.50 mL, 45.2 mmol, 1.50 equiv.) at 0° C. The mixture was stirred 1 hour at room temperature and was then heated to 80° C. The compound of formula VIII (2.50 mL, 30.2 mmol) was then added drop-wise to the reaction, and the resulting mixture was stirred 3 hours at 95° C. The reaction was then quenched by slow addition to ice (40 g). The pH of the resulting solution was 2, and it was raised to 5 by slow addition of 12N aqueous sodium hydroxide solution (11.2 mL). The resulting aqueous solution was extracted with dichloromethane and/or ether (7×40 mL), and additional sodium hydroxide was added during extraction, as needed, to maintain a pH of 5. The extracts were then combined, dried over sodium sulfate, filtered and concentrated to yield a brown oil (3.79 g, 59% yield).

### Example 2

### Preparation of Compound of Formula XI from the Compound of Formula IX

Potassium tert-butoxide (23.47 g of 95%, 199.1 mmol, 2.44 equiv.) was suspended in anhydrous DME (90 mL) and cooled to −60° C. TosMIC (23.76 g, 121.7 mmol, 1.49 equiv.) was dissolved in anhydrous DME (75 mL), and this was added drop-wise to the potassium tert-butoxide solution over 20 minutes. After stirring for 20 minutes between −60 and −55° C., the compound of formula IX, in anhydrous DME (55 mL), was added over 23 minutes. The reaction was stirred one hour at −55 to −50° C. to yield a thick suspension. Methanol (90 mL) was then added resulting in a clear brown solution. The cooling bath was removed, and after stirring 5 minutes in air, the reaction flask was immersed in an oil bath preheated to 85° C. The reaction was stirred for 1 hour. After cooling, the mixture was concentrated and the resulting tan solid was dissolved in water (180 mL) with acetic acid (9 mL). This was extracted with ethyl acetate (3×250 mL), and these extracts were combined, washed with saturated aqueous sodium chloride (100 mL), dried over sodium sulfate, filtered and concentrated to yield a brown oil (13.71 g). This oil was dissolved in dichloromethane and purified by silica-gel chromatography using a gradient from 0% to 15% dichloromethane-acetone to yield the compound of formula XI as a bright yellow oil (7.89 g, 63% yield).

### Example 3

### Preparation of Compound of Formula X from the Compound of Formula IX

The compound of formula IX (3.48 g, 31.6 mmol) was dissolved in methanol (25 mL) and sodium borohydride (2.50 g, 66.1 mmol, 2.09 equiv.) was added portion-wise with vigorous gas evolution. After stirring for 3 hours at room temperature, the reaction was cooled to 0° C. and slowly acidified to pH ˜1 with 4N aqueous hydrochloric acid (20 mL) over 55 minutes. A thick white slurry formed and this was stirred one hour at room temperature. The reaction was then basified by the gradual addition of saturated aqueous potassium carbonate solution (53.4 wt % K₂CO₃, 6.04 M; 10 mL). This resulted in a clear, colorless solution (pH=11), which was diluted with additional saturated potassium carbonate solution (200 mL) and was extracted with ethyl acetate (2×200 mL). The ethyl acetate extracts were combined, dried over sodium sulfate, filtered and concentrated to yield the compound of formula X as a light yellow oil (3.44 g, 97% yield).

### Example 4

### Preparation of Compound of Formula XI from the Compound of Formula X

The compound of formula X (0.201 g, 1.80 mmol) was dissolved in anhydrous dichloromethane (3 mL) and thionyl chloride (0.140 mL, 1.92 mmol, 1.06 equiv.) was added drop-wise at 0° C. The reaction was stirred 1 hour at 0° C. and then 1 hour at room temperature. The reaction was then concentrated at 50° C. and dried under vacuum for 2 hours to yield a white solid (0.286 g). This solid was suspended in anhydrous acetonitrile and triethylamine (0.750 mL, 5.38 mmol, 2.99 equiv.) was added. After stirring for a few minutes, tetraethylammonium cyanide (1.08 g, 6.90 mmol, 3.84 equiv.) was added in one portion. The reaction was stirred 18 hours at room temperature and was then diluted with water (15 mL) and extracted with ethyl acetate (3×20 mL). The extracts were combined, washed with saturated aqueous sodium chloride, dried over sodium sulfate, filtered and concentrated to yield a yellow oil (0.128 g). Purification by chromatography, eluting with 5% methanol-dichloromethane, yielded the compound of formula XI as a yellow oil (0.063 g, 30% yield).

### Example 5

### Preparation of Compound of Formula XII from the Compound of Formula XI

The compound of formula XI (10.49 g, 86:68 mmol) and ethyl formate (15.15 mL, 187.5 mmol, 2.16 equiv.) were dissolved in anhydrous DME (25 mL) and added drop-wise to a suspension of potassium-tert-butoxide (17.01 g of 95%, 144.3 mmol, 1.66 equiv.) in anhydrous DME (90 mL) in an open pressure tube. After addition was complete, the tube was sealed and stirred at 85° C. for 29 hours. After cooling, the resulting thick suspension was diluted with water (400 mL) to yield a solution of pH 8, and was extracted with ethyl acetate (3×400 mL). The aqueous solution was then acidified to pH 4 with concentrated hydrochloric acid (11 mL) resulting in the formation of a white precipitate. This suspension was then filtered, and the recovered solid was washed with water and dried under vacuum to yield the compound of formula XII as an off-white solid (11.66 g, 90% yield). Extraction of the aqueous filtrate with ethyl acetate (2×500 mL), followed by washing with brine, drying with sodium sulfate, filtering and concentrating resulted in an additional quantity of the product (0.74 g, 5% yield).

### Example 6

### Preparation of Compound of Formula IA from the Compound of Formula XII

The compound of formula XII (11.58 g, 77.72 mmol) was suspended in absolute ethanol (400 mL), and hydrazine monohydrochloride (10.67 g, 156.0 mmol, 2.00 equiv.) was then added. The mixture was stirred 15 hours at 90° C. to yield an orange suspension. After briefly allowing the reaction to cool, 7N NH₃ in methanol (25 mL) was added and the mixture was stirred for 20 minutes. The mixture was filtered to remove the precipitated solid (ammonium chloride). The filtrate was then concentrated to yield a light yellow solid, which was then loaded dry on a chromatography column and purified eluting with 10% methanol-dichloromethane followed by 10% 7N NH₃ in methanol-dichloromethane to yield the compound of formula IA as an off-white solid (11.35 g, 90% yield). ¹H NMR (400 MHz, DMSO-d₆): δ 11.4 (s, 1H), 7.76 (s, 1H), 7.54 (s, 1H), 7.48 (s, 1H), 4.54 (s, 2H), 3.79 (s, 3H). MS (MH⁺): 164.

Each and every reference (e.g., patent publications, issued patents, or scientific journal publications) mentioned in this patent application is incorporated herein by reference in its entirety for all purposes.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A process for preparing a compound of formula (I) 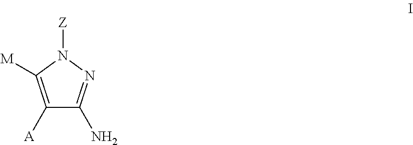
comprising:
(a) Converting a compound of formula II
A-H II
to a compound of formula III 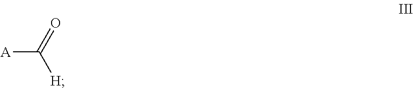
and either:
(b)(1) reacting the compound of formula III with the anion of tosylmethylisocyanide (TosMIC) to yield a compound of formula IV 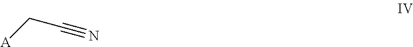
or:
(b)(2) Reducing the compound of formula III to the compound of formula V, 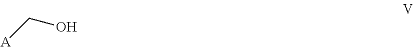
followed by conversion of the compound of formula V to the compound of formula IV; and thereafter:
(c) Acylating the compound of formula IV to a compound of formula VI; 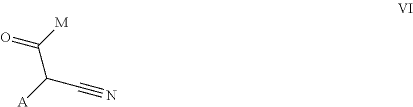
and
(d) Treating the compound of formula VI with a compound of formula VII, 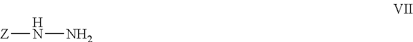
or one of its salts or hydrates, to yield the compound of formula I;
wherein:
A is selected from the group consisting of alkyl, cycloalkyl, aryl, and heteroaryl, each of which is independently unsubstituted or substituted with at least one W moiety;
M and Z are independently selected from the group consisting of H, alkyl, aralkyl, cycloalkyl, heterocyclyl, aryl and heteroaryl wherein each of said alkyl, aralkyl, cycloalkyl, heterocyclyl, aryl and heteroaryl is independently unsubstituted or substituted with at least one W moiety;
W is selected from the group consisting of alkyl, halo, cycloalkyl, heterocyclyl, aryl and heteroaryl.

2. The process of claim 1, wherein A is heteroaryl.

3. The process of claim 2, wherein A is 1-methyl-4-pyrazolyl.

4. The process of claim 1, wherein M is H.

5. The process of claim 1, wherein Z is H.

6. The process of claim 1, wherein the compound of formula I is

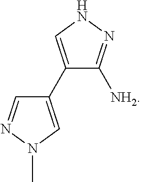

7. The process of claim 1, wherein in step (a), the conversion of the compound of formula II to the compound of formula III is carried out in the presence of a mixture of phosphorus oxychloride (POCl₃) and dimethylformamide.

8. The process of claim 1, wherein in step (b)(1), the anion of TosMIC is to prepared by treating TosMIC with a basic compound.

9. The process of claim 8, wherein said treatment is carried out at a temperature of about −78° C. to about −20° C.

10. The process of claim 8, wherein said basic compound is a metal hydroxide, metal oxide or metal alkoxide.

11. The process of claim 10, wherein said metal is an alkali or alkali earth metal.

12. The process of claim 10, wherein said basic compound is potassium tert-butoxide.

13. The process of claim 8, wherein said treatment is carried out in the presence of 1,2-dimethoxyethane (DME).

14. The process of claim 1, wherein in step (b)(1), the reaction of compound of formula III with the anion of TosMIC is carried out by stirring a mixture of the anion of TosMIC and the compound of formula III in a mixture of DME at a temperature of about −60° C. to about −50° C., followed by the addition of methanol, and heating of the mixture at a temperature of about 50° C. to the reflux temperature of the solvent mixture.

15. The process of claim 1, wherein in step (b)(2), the reduction of the compound of formula III to the compound of formula IV is carried out using sodium borohydride (NaBH₄).

16. The process of claim 1, wherein in step (b)(2), the conversion of the compound of formula V to the compound of formula IV is carried out in a two-step process involving in-situ conversion of the compound of formula V to the compound of formula V-A 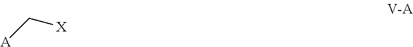
followed by conversion of the compound of formula V-A to the compound of formula IV, wherein in formula V-A, X is selected from the group consisting of halo, —S(O)₂alkyl, —S(O)₂aryl.

17. The process of claim 16, wherein in Formula V-A, X is Cl.

18. The process of claim 17, wherein the in-situ conversion of the compound of formula V to the compound of formula V-A is carried out using thionyl chloride (SOCl₂)

19. The process of claim 16, wherein the conversion of the compound of formula V-A to formula IV is carried out using tetraethylammonium cyanide.

20. The process of claim 1, wherein in step (c), the acylation of the compound of formula IV to the compound of formula VI is carried out using a basic compound and an acylating agent that is an alkyl ester of the acid MCOOH.

21. The process of claim 20, wherein said acylating agent is an ethyl, methyl, or isopropyl ester of formic, acetic, or benzoic acid.

22. The process of claim 21, wherein said acylating agent is ethyl formate.

23. The process of claim 20, wherein said basic compound is a metal hydroxide or metal alkoxide.

24. The process of claim 23, wherein said basic compound is potassium tert-butoxide.

25. The process of claim 1, wherein in step (d), the compound of formula VII is treated with a compound selected from the group consisting of hydrazine, hydrazine hydrate, and hydrazine salt.

26. The process of claim 25, wherein the hydrazine salt is selected from the group consisting of hydrazine acetate, hydrazine hydrochloride, and hydrazine sulfate.

27. The process of claim 25, wherein in step (d), the compound of formula VI is condensed with hydrazine monohydrochloride to yield the compound of formula I.
